(19) United States
(12) Patent Application Publication
(10) Pub. No.: US 2011/0295027 A1
(43) Pub. Date: Dec. 1, 2011

(54) Catalytic Isomerisation of Fatty Acid Esters

(75) Inventors: Paul Ratnasamy, Louisville, KY (US); Moises A. Carreon, Louisville, KY (US)

(73) Assignee: UNIVERSITY OF LOUISVILLE RESEARCH FOUNDATION, INC., Louisville, KY (US)

(21) Appl. No.: 13/150,327

(22) Filed: Jun. 1, 2011

Related U.S. Application Data
(60) Provisional application No. 61/350,238, filed on Jun. 1, 2010.

(51) Int. Cl. C07C 67/333; C07C 67/48; C07C 69/00

(52) U.S. Cl. 554/126; 554/163; 554/1

(57) ABSTRACT

Catalytic isomerisation of fatty acid esters to form branched fatty acid esters comprises the steps of contacting a feedstock having at least one fatty acid ester with a catalyst and isolating from other reaction products branched fatty acid esters that are produced. The catalyst is a crystalline porous silicoaluminophosphate catalyst having silicon, aluminum, and phosphorous atoms in lattice framework positions. The branched fatty acid esters formed thereby demonstrate properties suitable for use in various applications.

### CROSS REFERENCE TO RELATED U.S. APPLICATION

This application claims priority to U.S. Provisional Application No. 61/350,238 filed on Jun. 1, 2010.

### FIELD OF THE INVENTION

The invention relates to fatty acid derivatives comprising at least one fatty acid ester that undergoes catalytic isomerisation producing branched isomers having different properties compared to pre-isomerisation starting materials, which are used in various applications, such as biofuels, biolubricants, and deicing agents.

### BACKGROUND OF THE INVENTION

Uses of fatty acid esters as biofuels, biolubricants, and deicing agents are known in the art. For example, a variety of biofuels serve as alternatives to petroleum-based transportation fuels. Biodiesel, which contains unsaturated, linear fatty acid methyl esters, is a non-limiting example of such an alternative biofuel. The branched isomers of linear fatty acid esters provide some properties that differ from the properties of linear analogs. In some applications, the lower melting point of the branched isomers provides improved cold flow properties, such as lower melting points, cloud points, and pour points, compared to linear analogs.

Various means have been used to carry out isomerisation of fatty acid ester starting materials, for the purpose of producing branched isomers with different or improved properties. These include contacting a feedstock comprising at least one fatty acid ester with a catalyst at relatively high temperature and/or pressure to carry out the isomerisation reaction. Solid catalysts comprising aluminosilicate (zeolitic) frameworks have been used in such applications.

### SUMMARY OF INVENTION

As described and claimed herein, catalytic isomerisation of fatty acid esters uses highly ordered molecular structures known as silicoaluminophosphates as a catalyst in the production of branched isomers from fatty acid esters, which provide improved properties compared to the starting materials. In some embodiments, the isomers have one or more alkyl side chains. The improved properties are associated with the breaking and rearrangement of chemical bonds of the starting material molecules, and include (as non-limiting examples) reduced viscosity, lower melting point, and increased oxidative stability of the products.

In some embodiments, the acidity of the active sites of some silicoaluminophosphate catalysts promotes the formation of carbenium ions in the transition state, thus increasing the rate of reaction producing branched products. Also, the formation of products by hydrolysis, cracking, transalkylation, and transesterification is limited for reactions carried out over these catalysts, compared to more conventional processes for converting linear fatty acid esters to branched fatty acid esters. In some embodiments, silicoaluminophosphate catalysts are relatively reusable because they are can be regenerated through methods that are known to those of ordinary skill in the relevant art.

In some embodiments, starting materials are contained in a feedstock comprising fatty acid esters having one or more conjugated double bonds. Catalytic isomerisation of such fatty acid esters yields branched fatty acid esters having the same number of isomerised double bonds, wherein at least one pair of the conjugated double bonds is converted to unconjugated double bonds in the products. Compared to linear fatty acid ester starting materials, these products have higher chemical stability against oxidation, in addition to reduced pour points, cloud points, and cold filter plug points.

### DESCRIPTION OF MULTIPLE EMBODIMENTS AND ALTERNATIVES

Catalytic isomerisation of fatty acid esters converts linear fatty acid esters, to branched fatty acid esters having at least one alkyl side chain. In some embodiments, the at least one alkyl side chain is a C1-C4 aliphatic side chain. Various fatty acid esters are converted by the process, such as for example, fatty acid methyl esters. In some embodiments, catalytic isomerisation of fatty acid esters yields fatty acid esters having between approximately one and three alkyl side chains, although the embodiments described herein are not limited to products having three or fewer side chains.

In some embodiments, the fatty acid esters are unsaturated fatty acids esters with one or more double bonds. For fatty acid ester starting materials containing more than one double bond, the bonds are conjugated or, alternatively, unconjugated double bonds. In some embodiments, catalytic isomerisation of fatty acid esters converts at least one pair of conjugated double bonds of fatty acid esters to unconjugated double bonds.

Catalytic isomerisation of fatty acid esters is performed over a catalyst that is a crystalline, silicoaluminophosphate molecular sieve having silicon, aluminum, and phosphorous atoms arranged in lattice framework positions, sometimes referred to as “SAPO.” In some embodiments, the starting materials are comprised of a feedstock that includes fatty acid alkyl esters, such as for example, fatty acid methyl esters.

Isomerized fatty acid esters exhibit different cold flow properties compared to analogous linear fatty acid esters. Cold flow properties include pour point, cloud point, and cold filter plug point of fatty acid esters, as defined according to common and ordinary meanings in the art for those terms. In general, catalytic isomerisation of fatty acid esters yields branched fatty acid esters with reduced pour points, cloud points, and cold filter plug points compared to the analogous linear isomers. This lessens or eliminates the need for fuel additives such as pour point depressants and viscosity modifiers, as are used with some petroleum-based fuels.

In some embodiments, starting materials are contained in a feedstock comprising fatty acid esters having one or more conjugated double bonds. Catalytic isomerisation of such fatty acid esters yields branched fatty acid esters having one or more isomerised double bonds, wherein at least one pair of the conjugated double bonds is converted to unconjugated double bonds in the products. By comparison to the analogous linear fatty acid esters having a greater number of conjugated double bonds, the products exhibit higher chemical stability against oxidation, in addition to reduced pour points, cloud points, and cold filter plug points.

Fatty acid ester starting materials are obtained from a number of processes that are known in the art, such as for example, transesterification of triglyceride oils and fats. By way of illustration, not limitation, transesterification of soybean oil with methanol in the presence of alkali catalysts produces a mixture of fatty acid methyl esters such as, for example, methyl oleate, methyl linoleate, methyl linolenate, methyl stearate, and methyl palmitate. Methyl linoleate and methyl linolenate are fatty acid methyl esters having at least one pair of conjugated double bonds. Other starting materials suitable for catalytic isomerisation of fatty acid esters include food grade vegetable oils, such as for example, corn, soy, and canola and other sources, including but not limited to animal fats, grease, used oils and other waste products.

In some embodiments, catalytic isomerisation of fatty acid esters comprises contacting linear, unsaturated fatty acid esters with silicoaluminophosphate catalysts having catalytic sites that are acidic, at temperatures in a range from approximately 100° C. to 400° C., and pressures from approximately atmospheric to 50 bar, and isolating branched isomer products. Optionally, the isomerisation occurs in single batch mode. Alternatively, the isomerisation occurs in continuous mode, such as for example, in a fixed bed reactor or flow reactor. Alternatively, the isomerisation occurs utilizing modes of catalytic reactions of hydrocarbons that are known in the art.

Silicoaluminophosphate catalysts are synthesized hydrothermally at temperatures in a range from approximately 100° C. to 200° C. from various templates known in the art, such as for example organic amine templates, quaternary ammonium templates, and alkyl amines. In some embodiments the organic template is di-n-propylamine. Sources for reactive alumina, phosphate, and silica include, but are not limited to hydrated aluminum oxide, phosphoric acid, and silica sol.

In some embodiments, the silicoaluminophosphate catalysts in the anhydrous form are represented by the formula (SiₓAl_{y}P_{z})O₂, where x, y, and z represent mole fractions of silicon, aluminum, and phosphorus; the sum of x, y, and z is one; and x is in a range from approximately 0.01 to 0.98, y is in a range from approximately 0.01 to 0.60, and z is in a range from approximately 0.01 to 0.52. Template species retained within the products are removed through calcination at temperatures in a range sufficient to free the intracrystalline voids and to provide pores into which starting materials are adsorbed. In some embodiments, the temperatures are in a range from approximately 400° C. to 600° C.

The catalytic sites on the silicoaluminophosphate catalysts are located in uniform pores comprising tetrahedral oxide frameworks of silicon, aluminum, and phosphorus. The volumes of the pores are in a range from approximately 0.18 cm³/g to 0.48 cm³/g, as determined by techniques known in the art, such as for example, McBain-Baker gravimetric techniques. The pore diameters are in a range from approximately 0.3 nanometers (nm) to 0.8 nm. Silicoaluminophosphate catalysts, having any one of various pore dimensions selected by a user, promote size-selective and shape-selective separations. In some embodiments, the pores comprise 6- to 12-membered rings. By way of illustration, not limitation, some 10-membered rings have an elliptical cross-section of approximately 0.39 nm to 0.63 nm with ten tetrahedral atoms forming the framework. In some embodiments, pore openings are in the form of puckered rings. Compared to circular rings or elliptical rings, puckered rings spatially constrain the size and geometry of molecules the pores can hold.

In various alternative embodiments, the ratio of silicon to aluminum and phosphorous within the framework produces an overall negative charge localized within the catalytic sites which, combined with cations and hydroxyl groups dispersed superficially, provide a range of charge-selectivity during catalytic isomerisation, while the volume and diameter of the pores influences shape-selectivity. Examples of small pore molecular sieves include 6-membered rings, such as for example, SAPO-16 and SAPO-20, which admit only small molecules (e.g., water and ammonia). Silicoaluminophosphate catalysts with larger pores include those with 8-membered rings having pore openings of approximately 4.3 Å, such as for example, SAPO-17, SAPO-34, SAPO-35, SAPO-42 and SAPO-44. Silicoaluminophosphate catalysts having 10-membered rings or puckered 12-membered rings, such as for example, SAPO-11, SAPO-31, SAPO-40, and SAPO-41, have pore diameters which are in a range from approximately 6.2 Å to 7.8 Å. SAPO-5 and SAPO-37 have pore structures with circular 12-membered ring openings.

In some embodiments, catalytic isomerisation of fatty acid esters is carried out over a catalyst having a plurality of pores formed within a 10-membered ring framework, such as for example, SAPO-11. SAPO-11 is a member of the family of silicoaluminophosphate catalysts having one-dimensional, 10-membered rings with pore diameters in a range from approximately 3.5 Å to 6.5 Å, with each pore having a uniform elliptical cross-section of approximately 0.39 nm to 0.63 nm. The organic template used in synthesis is removed by calcination conducted at approximately 550° C., which provides intracrystalline porous volumes of approximately 0.18 ml/g. While SAPO-11 has stronger acidity compared to some other silicoaluminophosphate catalysts, due to the ratio of framework silicon atoms, other silicoaluminophosphate catalysts with moderate acidity are also sufficient. Optionally, the ratio of silicon within the framework is modified by various techniques known in the art, such as for example, dealuminating following synthesis.

In some embodiments, the silicoaluminophosphate catalysts have pores containing catalytic sites that are sufficiently acidic to provide charge-selectivity for the isomerisation of fatty acid esters into branched fatty acid esters. Shape selective, 10-membered ring pores in the catalyst increase desirable selectivity toward branched products, and inhibit cracking and oligomerisation reactions that would result in less desirable products. Larger crystals having longer 10-membered ring pores have higher shape selective catalytic properties. Such crystals can be obtained by proper control of catalyst preparation conditions including temperature, concentrations, duration of crystallization etc. well known to those in the art. Pore sizes in the range of approximately 0.39 nm to 0.63 nm suppress formation of products containing numerous branches, or overly long branches, thereby limiting formation of less desirable products such as those susceptible to hydrocracking For example, for pores with diameters no greater than about 0.63 nm, the products tend to have a higher concentration of fatty acid methyl esters, whereas the concentration of ethyl- and propyl-branched products is comparatively higher for catalysts having larger diameters or greater volumes. By way of further illustration, isomerisation of n-decane over silicoaluminophosphate catalysts having a 10-membered ring framework is expected to produce a lower concentration of di- and tri-branched products, than isomerisation of n-decane carried out over silicoaluminophosphate catalysts having a 12-membered ring framework.

In some embodiments, the acidity of the active sites promotes the formation of carbenium ions in the transition state, thus increasing the rate of reaction leading to branched products, and promoting double bond isomerisation within the branched products. Compared to the linear fatty acid ester starting materials, these branched fatty acid ester products have at least one fewer pair of unconjugated double bonds, and the formation of products by hydrolysis, cracking, transalkylation, and transesterification is limited. Moreover, silicoaluminophosphate catalysts are relatively reusable or capable of regeneration through methods that include combustion of the carbonaceous deposits in air.

In some embodiments, catalytic isomerisation of fatty acid esters uses fatty acid ester starting materials, which are represented by the chemical formula R-COOR₁. These are of various chain lengths, such as for example, wherein R ranges from approximately 10 carbons to 24 carbons. Generally, R₁ is chosen from the group methyl, ethyl, propyl, and butyl. Reaction temperatures are in a range from approximately 100° C. to 400° C., or, more specifically, in a range from approximately 250° C. to 350° C., and at pressures ranging from approximately atmospheric to 50 bar.

In some embodiments, such as for example, reactions occurring in batch mode, the weight of solid catalyst to fatty acid methyl ester starting material (i.e., when R₁ is methyl) is in a range from approximately 0.1 wt. % to 5.0 wt. %. For reactions occurring in continuous mode, such as for example, in a fixed bed reactor, liquid hourly space velocity of the fatty acid methyl ester feedstock varies from 0.1-4.0 ml/ml catalyst/hr. Liquid hourly space velocity is the volume (in ml) of fatty acid ester passed thru an unit volume of catalyst (in ml) of catalyst bed per hour. In some embodiments, catalytic isomerisation of fatty acid esters is conducted in the presence of a carrier gas, such as for example, nitrogen or hydrogen.

It is understood by those of ordinary skill in the art that adjustments to reaction conditions influence the rate of conversion of fatty acid esters and the selectivity of the reaction toward branched isomers, both with and without isomerisation of conjugated double bonds. Accordingly, the description of the reaction conditions herein is meant as illustrative, and not limiting. Additionally, the following examples further illustrate catalytic isomerisation of fatty acid esters. These examples are non-limiting and merely characteristic of multiple alternative embodiments as described and claimed herein.

### Example 1

### Catalytic Isomerisation of Methyl Oleates over SAPO-11, compared to five other catalysts (1A-1E)

The activity and selectivity of SAPO-11 were compared to five zeolite catalysts, which are listed as 1A-1E in Table 1. Each catalyst in this example was prepared according to conventional processes known in the art. SAPO-11 was in the form of extrudates having a diameter of approximately 1 mm and having a length of approximately 3 mm, while the other catalysts were in powder form. Table 1 provides some of the physicochemical properties of the solid catalysts.

For each reaction, 0.5 grams of catalyst and 5 ml of methyl oleate (both cis- and trans isomers) were loaded into a reactor, which was a Teflon-lined, stainless steel autoclave (20 ml).

The weight percentage of aluminum, phosphorous, and silicon contained in the SAPO-11 catalyst was approximately 23.7%, 21.3%, and 2.6%, respectively.

TABLE 1

| | | SiO₂/Al₂O₃ | Na₂O | Surface area | Relative |
|---|---|---|---|---|---|
| | Catalyst | (mol) | (% wt) | (BET)m²/g | Acidity* |
| 1 | SAPO-11 | (as stated above) | 0.016 | 253 | 1.0 |
| 1A | H-ZSM-5 | 30.1 | 0.01 | 361 | 1.3 |
| 1B | H-ZSM-5 | 80.7 | 0.02 | 403 | 1.5 |
| 1C | H-Beta | 23.6 | 0.05 | 510 | 1.5 |
| 1D | H-Y | 5.1 | 0.9 | 660 | 1.5 |
| 1E | MCM-22 | 26.0 | 0.034 | 530 | 2.2 |

*moles of dipropylamine adsorbed/gm of dry catalyst (×10⁻⁸)

The feedstock containing methyl oleates underwent catalytic isomerisation of fatty acid esters over each of the catalysts identified in Table 1. Under autogeneous pressure, the reactor was heated to 200° C. Each reaction continued under the above-described conditions for 15 h, until terminated by cooling the reactor to 25° C. All the reaction products consisted of a single liquid layer. The catalyst was separated from the liquid layer by filtration. However, with these examples separation can alternatively be performed through distillation or other separation methods known in the art.

The liquid layer was analyzed by a combination of gas chromatography, mass spectrometry, proton NMR and ¹³C NMR. The pH of the reaction mixture did not change after the reaction, nor did testing detect free fatty acid or methanol in the product, indicating that hydrolysis of methyl oleate to oleic acid and methanol did not occur. The liquid layer consisted of skeletally isomerised branched methyl oleates, cracked (mainly C16) esters and linear monounsaturated isomers having at least one double bond at a different location compared to the starting materials, indicating occurrences of double bond isomerisation. Concentrations of branched products were estimated based upon changes in the peak-intensities of NMR spectra. Table 2 quantifies the conversion of methyl oleate to branched isomers, as well as the selectivity for the formation of branched skeletal isomers of methyl oleate, associated with each catalyst.

TABLE 2

| | | Methyl | Selectivity |
|---|---|---|---|
| | | Oleate | for |
| | | conversion | branched |
| | Catalyst | (wt %) | isomers(%) |
| Example 1 | SAPO-11 | 23 | 62 |
| Comparative: 1A | H-ZSM-5(30) | 6 | 35 |
| Comparative: 1B | H-ZSM-5(80) | 2 | 34 |
| Comparative: 1C | H-Beta | 45 | 23 |
| Comparative: 1D | H-Y | 62 | 19 |
| Comparative: 1E | MCM-22 | 38 | 4 |

### Example 2

### Catalytic Isomerisation of commercial biodiesel feedstock over SAPO-11, compared to five other catalysts (2A-2E)

Commercial biodiesel feedstock underwent catalytic isomerisation of fatty acid esters over SAPO-11 and the five comparative catalysts (1A-1E) listed in Table 1. The feedstock was obtained by transesterification of waste oils with methanol using alkali catalysts according to processes known in the art, and otherwise meeting ASTM D 6751 specifications. Methyl oleate, methyl linoleate, methyl palmitate, methyl stearate and methyl linolinate were the major constituents of this sample of biodiesel.

As with Example 1, 0.5 grams of catalyst and 5 ml of feedstock were loaded into the reactor, a Teflon-lined, stainless steel autoclave (20 ml). Under autogeneous pressure, the reactor was heated to 200° C. Each reaction continued under the above-described conditions for 15 h, until terminated by cooling the reactor to 25° C. All the reaction products consisted of a single liquid layer. The catalyst was separated from the liquid layer by filtration.

Referring now to Table 3, the liquid product was analyzed by a combination of gas chromatography, mass spectrometry, proton NMR and ¹³C NMR as with Example 1. There was no change in the acid number of the reaction mixture after the reaction and no methanol was detected in the product, indicating that hydrolysis of the ester to acid and alcohol did not occur. Table 3 quantifies the selectivity for the conversion of feedstock into branched isomers from catalytic isomerisation of biodiesel over SAPO-11, and compares these results to the five other solid catalysts that were used.

TABLE 3

| | | Biodiesel | Selectivity |
|---|---|---|---|
| | | conversion | for skeletal |
| | Catalyst | (wt %) | isomers (%) |
| Example 2 | SAPO-11 | 5.9 | 44 |
| Comparative: 2A | H-ZSM-5(30) | ~0 | — |
| Comparative: 2B | H-ZSM-5(80) | ~0 | — |
| Comparative: 2C | H-Beta | 16.6 | 27 |
| Comparative: 2D | H-Y | 30.2 | 18 |
| Comparative: 2E | MCM-22 | 32.7 | 22 |

### Example 3

### Catalytic stability of SAPO-11 over time

10 gm of Sapo-11 extrudates as described in Example 1 were loaded in a continuous, fixed bed, downflow reactor. Methyl oleate feedstock was passed over the catalyst at a flow rate of 0.5 gm/gm of catalyst/hr at 250° C. at atmospheric pressure. Products were collected at various time-on-stream (TOS) values, then analyzed as described in Example 1. The results are provided in Table 4.

TABLE 4

| Time-on- | Methyl oleate | Selectivity for |
|---|---|---|
| stream | conversion | branched |
| (hrs) | (wt %) | isomers (%) |
| 5 | 18 | 71 |
| 20 | 22 | 70 |
| 32 | 21 | 68 |
| 52 | 24 | 70 |
| 71 | 25 | 69 |
| 98 | 22 | 67 |

### Example 4

### Conversion and selectivity for isomerisation over SAPO-11 as a function of temperature

Catalytic isomerisation of methyl oleate was carried out at five different temperatures in a stainless steel, Parr autoclave provided with a stirrer. For each reaction, 1 gm of Sapo-11 catalyst and 10 ml of methyl oleate reactant were loaded in the reactor. Sapo-11 was in the form of extrudates as described for Example 1. Under autogeneous pressure, the reactor was heated to various temperatures in the range of 200-300° C., as listed in Table 5. Each reaction was continued under the above-described conditions for 15 h, until terminated by cooling the reactor to 25° C. All the reaction products consisted of a single liquid layer. The catalyst was separated from the liquid layer by filtration. The liquid layer was analyzed as described in Example 1. The results are provided in Table 5.

TABLE 5

| Temperature | Methyl oleate | Selectivity for branched |
|---|---|---|
| (° C.) | conversion(wt %) | isomers(%) |
| 200 | 26 | 62 |
| 225 | 29 | 62 |
| 250 | 33 | 60 |
| 275 | 41 | 57 |
| 300 | 48 | 53 |

It is to be understood that the embodiments described herein are not limited in their application to the details of the steps set forth in the above descriptions and teachings or as illustrated in the above examples. Rather, catalytic isomerisation of fatty acid esters is capable of other embodiments and of being practiced or of being carried out in various ways, which would be within the scope of the coverages sought herein.

Also, it is to be understood that the phraseology and terminology used herein is for the purpose of description and should not be regarded as limiting. The use herein of “including,” “comprising,” “e.g.,” “such as,” “for example,” “containing,” or “having” and variations of these words and phrases is meant in a non-limiting way to encompass the items listed thereafter, and equivalents of those, as well as additional items.

Accordingly, the foregoing descriptions of several embodiments and alternatives are meant to illustrate, rather than as limiting. The descriptions herein are not intended to be exhaustive or to limit as to the precise steps and/or forms disclosed. It will be understood by those having ordinary skill in the art that modifications and variations of the process are reasonably possible in light of the above teachings and descriptions. Accordingly, it is intended that the claims define the scope of coverage.

We claim:

1. A process for catalytic isomerisation of fatty acid esters to form branched fatty acid esters, comprising the steps of:
(a) contacting a feedstock having at least one fatty acid ester with a catalyst comprising a crystalline porous silicoaluminophosphate catalyst having silicon, aluminum, and phosphorous atoms in lattice framework positions; and
(b) isolating from other reaction products branched fatty acid esters that are produced,
wherein the at least one fatty acid ester has a chemical formula represented by R-COOR₁.

2. The process of claim 1, wherein each of the at least one fatty acid ester molecules has approximately 10-24 carbon atoms in R, and R₁ is chosen from the group methyl, ethyl, propyl, and butyl.

3. The process of claim 1, wherein at least one fatty acid ester in the starting material is linear, and at least one of the products comprises a fatty acid ester having at least one alkyl side chain bonded to a carbon on the R group.

4. The process of claim 3, wherein the at least one alkyl side chain contains 1-4 carbons.

5. The process of claim 1, wherein at least one fatty acid ester in the starting material contains at least one pair of conjugated double bonds, which is converted to a pair of unconjugated double bonds in a reaction product.

6. The process of claim 1, wherein the crystalline porous silicoaluminophosphate catalyst is a molecular sieve having an unidimensional channel pore structure.

7. The process of claim 1, wherein the chemical composition of the silicoaluminophosphate catalyst is represented by the formula (SiₓAl_{y}P_{z})O₂; wherein x, y, and z represent mole fractions of silicon, aluminum, and phosphorous; and wherein the sum of x, y, and z is approximately one.

8. The process of claim 7, wherein x is in a range from approximately 0.01 to 0.98, y is in a range from approximately 0.01 to 0.60, and z is in a range from approximately 0.01 to 0.52.

9. The process of claim 8, wherein the silicon content of the silicoaluminophosphate catalyst is at least approximately 2% by weight.

10. The process of claim 7, wherein the crystalline framework of the catalyst is SAPO-11.

11. The process of claim 7, wherein the crystalline framework of the catalyst is SAPO-34.

12. A process for catalytic isomerisation of fatty acid esters represented by the chemical formula R-COOR₁, to form branched fatty acid esters, comprising the steps of:
(a) contacting a feedstock having at least one fatty acid ester with a catalyst comprising a crystalline porous silicoaluminophosphate catalyst having silicon, aluminum, and phosphorous atoms in lattice framework positions; and
(b) isolating from other reaction products branched fatty acid esters that are produced,
wherein each of the at least one fatty acid ester molecules has approximately 10-24 carbon atoms in R; R₁ is chosen from the group methyl, ethyl, propyl, and butyl; at least one of the products comprises a fatty acid ester having at least one alkyl side chain bonded to a carbon on the R group; and the crystalline porous silicoaluminophosphate catalyst is a molecular sieve having an unidimensional channel pore structure; and the contacting step is carried out at a temperature no greater than approximately 350 ° C.

13. The process of claim 12, wherein the chemical composition of the silicoaluminophosphate catalyst is represented by the formula (SiₓAl_{y}P_{z})O₂; wherein x, y, and z represent mole fractions of silicon, aluminum, and phosphorous; and wherein the sum of x, y, and z is approximately one.

14. The process of claim 13, wherein x is in a range from approximately 0.01 to 0.98, y is in a range from approximately 0.01 to 0.60, and z is in a range from approximately 0.01 to 0.52 and the silicon content of the silicoaluminophosphate catalyst is at least approximately 2% by weight.

15. A mixture comprising at least one branched fatty acid ester represented by the chemical formula R-COOR₁, the at least one branched fatty acid ester being prepared by:
(a) contacting a feedstock comprising at least one fatty acid ester with a catalyst comprising a crystalline porous silicoaluminophosphate catalyst having silicon, aluminum, and phosphorous atoms in lattice framework positions; and
(b) isolating from other reaction products branched fatty acid esters that are produced,
wherein the at least one branched fatty acid ester has at least one more alkyl side chain than the fatty acid ester in the feedstock.

16. The process of claim 15, wherein R1 is chosen from the group methyl, ethyl, propyl, and butyl.

17. The process of claim 15, wherein each of the at least one fatty acid ester molecules has approximately 10-24 carbon atoms in R.

18. The process of claim 15, wherein the chemical composition of the silicoaluminophosphate catalyst is represented by the formula (SiₓAl_{y}P_{z})O₂; wherein x, y, and z represent mole fractions of silicon, aluminum, and phosphorous; and wherein the sum of x, y, and z is approximately one.

19. The process of claim 18, wherein x is in a range from approximately 0.01 to 0.98, y is in a range from approximately 0.01 to 0.60, and z is in a range from approximately 0.01 to 0.52.

20. The process of claim 19, wherein the silicon content of the silicoaluminophosphate catalyst is at least approximately 2% by weight.

21. The process of claim 18, wherein the crystalline framework of the catalyst is SAPO-11.

22. The process of claim 18, wherein the crystalline framework of the catalyst is SAPO-34.
